(19) United States
(12) Patent Application Publication
(10) Pub. No.: US 2019/0167736 A1
(43) Pub. Date: Jun. 6, 2019

(54) BACTERIOPHAGE FOR TREATING STAPHYLOCOCCUS INFECTIONS

(71) Applicant: Enbiotix, Inc., Cambridge, MA (US)

(72) Inventors: Daniel J. FERULLO, Cambridge, MA (US); Jeffrey A. RADDING, Cambridge, MA (US)

(21) Appl. No.: 15/746,663

(22) PCT Filed: Jul. 25, 2016
(86) PCT No.: PCT/US2016/043815

§ 371 Date: Jan. 22, 2018

Related U.S. Application Data
(60) Provisional application No. 62/343,209, filed on May 31, 2016.
(60) Provisional application No. 62/196,015, filed on Jul. 23, 2015.

(51) Int. Cl. A61K 35/76; A61P 31/04; C12N 7/00

(52) U.S. Cl. CPC A61K 35/76; A61P 31/04; C12N 2795/10221; C12N 2795/10232; C12N 7/00

(57) ABSTRACT

The present invention relates to GRCS bacteriophages, as well as to methods and compositions for the treatment of prosthetic joint infection. Particularly, the present invention provides bacteriophages specific against Staphylococcus aureus from prosthetic joint infections.

### PRIORITY

This application claims priority to U.S. Provisional Application No. 62/343,209 filed May 31, 2016, and U.S. Provisional Application No. 62/196,015 filed Jul. 23, 2015, each of which is hereby incorporated by reference in its entirety.

### FIELD OF THE INVENTION

The present invention relates to methods and compositions for the treatment of Staphylococcus infections, including prosthetic joint infections. Particularly, the present invention provides bacteriophages with high infectivity against Staphylococcus aureus in prosthetic joint infections.

### BACKGROUND OF THE INVENTION

There are millions of patients with prosthetic joints, including, for example, those with total hip prostheses and total knee prostheses. Due to the aging population and the increasing prevalence of obesity, the numbers of prosthetic joint surgeries are expected to increase each year. It is estimated that by 2020, 2.5 million individuals will undergo surgeries each year to insert a prosthetic joint or to replace an existing prosthetic joint.

Prosthetic joint infection (PJI) is a devastating complication of prosthetic joint surgeries. The incidence of PJI is about 1-2.5% for primary hip or knee replacements and about 2.1-5.8% for revision surgeries. Staphylococcus aureus causes 20-40% of prosthetic hip and knee arthroplasty infections, resulting in multiple surgeries, replacement of the prosthesis, long term antibiotic therapy, and potentially, arthrodesis, or amputation of the infected limb. Debridement, antibiotic therapy and implant retention (DAIR) is effective in selected patients with acute PJI. However, DAIR is not appropriate for those patients that do not meet the selection criteria and those with chronic PJI. In cases that are inappropriate for DAIR and in chronic cases, the presence of biofilms, among other things, is often an impediment to effective antibiotic therapy and patients therefore undergo either significant revision of the prosthetic joint, or 1-stage or 2-stage joint replacement, thus removing the biofilm by replacing the joint. This leads patients to endure multiple surgeries to treat their condition. Ineffectively treated or untreated PJI results in long-term functional handicap, risk of amputation, and even death.

Accordingly, there remains a need for effective treatment of prosthetic joint infections, including chronic PJI and PJI resulting from Staphylococcus aureus infection, particularly with respect to removal of the biofilm to enable effective antibiotic therapy and retention of the implant as an alternative to prosthetic joint replacement.

### SUMMARY OF THE INVENTION

The present invention provides bacteriophages with high infectivity particularly against Staphylococcus aureus in prosthetic joint infection (PJI). As demonstrated herein, a GRCS bacteriophage has high infectivity across clinical isolates from PJI, as compared to isolates from other clinical presentations. This result can be attributable at least in-part to the presence of bacteriophage gene 18505614 (a putative minor tail protein).

Accordingly, in one aspect, the present invention provides methods for treating PJI by administering to an infected area a GRCS bacteriophage, or a bacteriophage comprising the minor tail protein encoded by the GRCS bacteriophage gene 18505614, or a functional derivative thereof. The functional derivative may comprise an amino acid sequence that is at least 70% identical to the minor tail protein encoded by the GRCS bacteriophage gene 18505614. The minor tail protein or the functional derivative thereof may recognize a surface determinant on Staphylococcus aureus from prosthetic joint infections (PJIs). The bacteriophage can promote elimination of Staphylococcus aureus, and in some embodiments the bacteriophage can be engineered to promote clearance of other microbial agents in the infection and/or promote clearance of a biofilm associated with the PJI. Exemplary bacteriophage engineering strategies include expression of antimicrobial peptides at the infection site, expression of biofilm dispersing agents at the infection site, and/or the expression of one or more antibiotic potentiating genes at the infection site.

In another aspect, the present invention provides an engineered GRCS bacteriophage or a bacteriophage having a minor tail protein encoded by the GRCS bacteriophage gene 18505614, or a functional derivative thereof. The bacteriophage may be engineered to comprise genetic material encoding enzymes or polypeptides that promote clearance of a wide spectrum of microbial pathogens that may exist at the site of infection, such as antimicrobial peptides (AMPs) or lytic enzymes. In an embodiment, the bacteriophage is engineered to encode a biofilm-degrading enzyme that will be functionally expressed and optionally secreted by infected bacteria. In a further embodiment, the bacteriophage is engineered to encode for functional expression at the site of infection of at least one gene that increases the susceptibility of a bacterial cell to an antimicrobial agent, such as an inhibitor of an antibiotic resistance gene or inhibitor of a cell survival repair gene. The bacteriophage may be provided as a pharmaceutically-acceptable composition suitable for application to PJI's.

In various embodiments, the bacteriophage may be engineered to encode one or more markers whose expression will aid in detection of susceptible bacteria. In these aspects, the present invention further provides methods for detecting the presence of absence of susceptible Staphylococcus aureus in a sample derived from a subject having prosthetic joint infection, including the steps of exposing the sample to the bacteriophage and assaying the sample to detect the presence or absence of marker expression.

In other aspects, the invention provides a method for making GRCS phage from Staphylococcus host cells transformed with a bacterial artificial chromosome harboring the GRCS genome. Surprisingly, genomic dsDNA from GRCS phage is able to replicate in the absence of the phage terminal protein, allowing replication of the genome in E. coli, from which phage can be propagated upon transformation of the DNA into Staphylococcus host cells. Phage produced with this method are useful for treating infection involving Staphylococcus, including but not limited to PJI.

Other aspects and embodiments of the invention will be apparent from the following detailed description.

### DESCRIPTION OF THE DRAWINGS

FIG. 1 depicts a genome sequence comparison of three bacteriophages GRCS, P68, and 44AHJD. The arrow indicates the GRCS gene 18505614 which encodes for a putative minor tail protein.

FIGS. 2A and 2B show Podoviridae bacteriophage efficiency of plating (EOP) against Staphylococcus aureus from prosthetic joint infections. Lytic efficiency was scored visually with a score of 4 indicating complete clearing, 3 indicating clearing throughout but with faint turbidity through the cleared zone, 2 indicating substantial turbidity, 1 indicating a few individual plaques, and 0 indicating no clearing.

FIG. 3 shows Podoviridae bacteriophage efficiency of plating (EOP) against Staphylococcus aureus from other clinical isolates. Lytic efficiency was scored visually with a score of 4 indicating complete clearing, 3 indicating clearing throughout but with faint turbidity through the cleared zone, 2 indicating substantial turbidity, 1 indicating a few individual plaques, and 0 indicating no clearing.

FIG. 4 provides photographs of Podoviridae bacteriophage efficiency of plating (EOP) in overlay assays on lawns of Staphylococcus aureus from prosthetic joint infections.

FIG. 5 shows a dot-matrix homology between the major tail protein of GRCS compared to 3 other Podoviridiae phages that are highly related in sequence (A), and the same comparisons for the minor tail protein (B).

FIG. 6 illustrates assembly and introduction of the GRCS genome into a bacterial artificial chromosome (BAC). The construct may then be amplified in E. coli followed by transformation into a GRCS host strain.

FIG. 7 demonstrates that GRCS/BAC produces GRCS phage particles.

FIG. 8 illustrates gene insertion (GFP) into GRCS/BAC.

### DETAILED DESCRIPTION OF THE INVENTION

Prosthetic joint infection (PJI) is often characterized by the presence of Staphylococcus aureus and/or other microbial strains. These microbial organisms secrete numerous enzymes and toxins resulting in pain, inflammation, and other symptoms. Further still, these microbial organisms generate biofilms, which can protect the organisms from the host immune system and from antibiotics thus rendering prosthetic joint infections particularly difficult to treat. Although the etiology of PJI is complex, infections with S. aureus are especially difficult, due to the virulent nature of the bacteria and rapid biofilm formation.

The present invention provides bacteriophages with high infectivity particularly against Staphylococcus aureus in prosthetic joint infections. As demonstrated herein, GRCS bacteriophage has high infectivity across clinical isolates from PJI's, and not across isolates from other clinical presentations, a result which can be attributable at least in part to the presence of bacteriophage gene 18505614 (a putative minor tail protein) based on genomic analysis.

In one aspect, the present invention provides a method for treating prosthetic joint infections comprising administering to an infected area a GRCS bacteriophage or a bacteriophage having a minor tail protein encoded by the GRCS bacteriophage gene 18505614. In various embodiments, the bacteriophage is a Podoviridae bacteriophage. In an embodiment, the bacteriophage is a Podoviridae GRCS bacteriophage. In various embodiments, the bacteriophage is an engineered bacteriophage as described herein.

Many bacteriophages have been isolated that have Staphylococcus aureus as a natural host. See generally, Xia and Wolz, Phages of Staphylococcus aureus and their impact on host evolution, Infection, Genetics and Evolution 21:593-601 (2014). The GRCS bacteriophage was isolated from raw sewage collected from a treatment plant in India, and its complete genome sequence is known. Swift and Nelson, Complete Genome Sequence of Staphylococcus aureus Phage GRCS, Genome Announc. Vol. 2, Issue 2 (2014). GRCS is a lytic phage classified in the Podoviridae family. Phages of the Podoviridae family are characterized by having very short, noncontractile tails. Podoviridae viruses are non-enveloped, with icosahedral and head-tail geometries.

In various embodiments, the bacteriophage comprises the GRCS bacteriophage gene 18505614, which is believed to at least in-part provide the high infectivity rate against S. aureus isolated from PJI. Gene 18505614 comprises the nucleotide sequence of SEQ ID NO:1. Various derivatives can be created of Gene 18505614, including to optimize for expression and/or to encode variant proteins with enhanced and/or similar ability to provide for high infectivity rate of S. aureus PJI clinical isolates. In some embodiments, the bacteriophage comprises a nucleotide sequence encoding the minor tail protein (or derivative thereof as described below), and which may have at least about 40%, at least about 50%, at least about 60%, at least about 70%, at least about 80%, at least about 90%, at least about 95%, at least about 96%, at least about 97%, at least about 98%, or about 99% identity with SEQ ID NO: 1.

In various embodiments, the bacteriophage comprises genetic material encoding a minor tail protein, for example, as encoded by the GRCS bacteriophage gene 18505614. In some embodiments, the minor tail protein comprises the amino acid sequence of SEQ ID NO:2. In some embodiments, the bacteriophage comprises a functional derivative of the minor tail protein encoded by the GRCS bacteriophage gene 18505614. Without wishing to be bound by theory, it is believed that the minor tail protein encoded by the GRCS bacteriophage gene 18505614, or a functional derivative thereof, recognizes a surface determinant on Staphylococcus aureus associated with prosthetic joint infections.

In various embodiments, the functional derivative of the minor tail protein encoded by the GRCS bacteriophage gene 18505614 has an amino acid sequence that is at least about 60%, or at least about 65%, or at least about 70%, at least about 75%, at least about 80%, at least about 85%, at least about 90%, at least about 92%, at least about 95%, at least about 96%, at least about 97%, at least about 98%, or at least about 99% identical with SEQ ID NO:2. Functional derivatives can be determined by assaying for the spectrum of infectivity of phages that comprise the gene across Staphylococcus aureus isolates from PJI's, and/or isolates from other clinical presentations.

In various embodiments, the bacteriophage may comprise a protein having one or more amino acid mutations relative to the minor tail protein encoded by the GRCS bacteriophage gene 18505614. For example, the minor tail protein may have from 1 to about 20, or from 1 to about 15, or from 1 to about 10 amino acid mutations relative to the minor tail protein encoded by the GRCS bacteriophage gene 18505614. In some embodiments, the one or more amino acid mutations may be independently selected from substitutions, insertions, deletions, and truncations. In some embodiments, the amino acid mutations are amino acid substitutions and/or truncations.

In various embodiments, the bacteriophage is engineered to encode one or more additional enzymes or polypeptides, which when expressed by the target bacteria, enhance the effectiveness for clearing the infection. In various embodiments, the bacteriophage is engineered to comprise a nucleic acid encoding a biofilm-degrading enzyme, such that the enzyme is expressed and optionally secreted by infected bacteria. Biofilms are polymeric structures secreted by microbial organisms such as bacteria to protect the bacteria from various environmental attacks, such as, host defenses, antibiotics and disinfectants. Biofilms have a regulated lifecycle including attachment, maturation and dispersal phases. For example, initial attachment in Staph biofilm is generally mediated in part by protein-protein interactions, as S. aureus express receptors for a number of host plasma proteins including fibrin and fibrinogen. Staphylococcal biofilms are composed of three classes of molecules forming the extracellular polymeric substance; poly-beta-1,6-N-acetylglucosamine (PNAG), proteins including phenol soluble modulins, Staph protein A, and others, as well as extracellular DNA of both bacterial and host origin. Further, there are differences between S. aureus and S. epidermidis biofilms. For example, S. epidermidis RP62A biofilm is degraded by DspB enzyme, and not by proteinase K or bovine DNase I, whereas S. aureus biofilms are insensitive to DSPB, but degraded by proteinase K and DNase I.

Bacteria in biofilms can be tolerant to antibiotic therapy. Tolerance can be due to the inability of the antibiotic to achieve significant concentrations in the biofilm, coupled with the metabolic quiescence of some biofilm bacteria. Thus, biofilm associated infections, particularly on abiotic surfaces, are difficult to treat with standard antibiotic therapy.

Biofilms may be found on any surface, including, prosthetic joints. Biofilm-degrading enzymes degrade biofilm matrix polymers by inhibiting biofilm formation, detach established biofilm colonies, and render biofilm-forming cells sensitive to killing by antimicrobial agents.

Exemplary enzymes useful for breaking down biofilms include, but are not limited to, dispersin B, alginate lyase, amylase, carbohydrase, carboxypeptidase, catalase, cellulase, chitinase, cutinase, cyclodextrin glycosyltransferase, deoxyribonuclease, disaggregatase enzymes, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, polysaccharide depolymerase, proteolytic enzyme, ribonuclease, transglutaminase, xylanase, DNase I, or lyase. In some embodiments, the biofilm-degrading enzyme include cellulases, such as the glycosyl hydroxylase family of cellulases (e.g., glycosyl hydroxylase 5 family of enzymes also called cellulase A), polyglucosamine (PGA) depolymerases, and colonic acid depolymerases (e.g., 1,4-L-fucodise hydrolase), depolymerazing alginase, and DNase I. Additional biofilm-degrading enzymes are described, for example, in U.S. Pat. No. 8,153,119, which is hereby incorporated by reference in its entirety. In an embodiment, the bacteriophage is engineered to comprise a nucleic acid encoding Dispersin B, an enzyme that hydrolyzes β-1,6-N-acetyl-D-glucosamine. Examples of a Dispersin B gene are described, for example, in U.S. Pat. No. 8,153,119, which is hereby incorporated by reference in its entirety. In an embodiment, the Dispersin B gene comprises the nucleotide sequence of Dispersin B from Actinobacillus actinomycetemcomitans, as shown for example in SEQ ID NO:3, and/or comprises the amino acid sequence of SEQ ID NO:4, or functional variant thereof.

In various embodiments, the functional variant of the Dispersin B enzyme has an amino acid sequence that is at least about 70%, at least about 75%, at least about 80%, at least about 85%, at least about 90%, at least about 92%, at least about 95%, at least about 96%, at least about 97%, at least about 98%, or at least about 99% identical with SEQ ID NO:4. Functional variants can be determined by assaying for hydrolysis of β-1,6-N-acetyl-D-glucosamine. In various embodiments, the Dispersin B may have one or more amino acid mutations relative to SEQ ID NO:4. For example, the Dispersin B may have from 1 to about 20, or from 1 to about 15, or from 1 to about 10 amino acid mutations relative to SEQ ID NO:4. In some embodiments, the one or more amino acid mutations may be independently selected from substitutions, insertions, deletions, and truncations.

In various embodiments, the bacteriophage is engineered to comprise a nucleic acid encoding at least one antimicrobial polypeptide, such that the antimicrobial polypeptide is expressed and optionally secreted by host bacteria. In some embodiments, the antimicrobial polypeptide is an antimicrobial peptide. Antimicrobial peptides are also called host defense peptides and are produced by species ranging from bacteria, fungi, insects, frogs, and mammals as part of the innate immune response. In some embodiments, the antimicrobial peptide comprises about 10 to about 60 amino acids, or about 12 to about 50 amino acids.

In some embodiments, the antimicrobial peptide may include two or more positively charged residues provided by, for example, arginine or lysine, and a large proportion (e.g., greater than 50%) of hydrophobic residues. In some embodiments, the secondary structures of the antimicrobial peptides may be, for example, α-helical, 3-stranded (e.g., due to the presence of 2 or more disulfide bonds), 3-hairpin or loop (e.g., due to the presence of a single disulfide bond and/or cyclization of the peptide chain), and extended.

In an embodiment, the antimicrobial peptide may be an anionic peptide, for example, rich in glutamic and aspartic acids. In another embodiment, the antimicrobial peptide may be a linear cationic α-helical peptide, for example, lacking in cysteine. In a further embodiment, the antimicrobial peptide may be a cationic peptide enriched in specific amino acids. For example, the antimicrobial peptide may be rich in proline, arginine, phenylalanine, glycine, or tryptophan. In another embodiment, the antimicrobial peptide may be an anionic and cationic peptide that contains at least one cysteine and disulfide bond. For example, the antimicrobial peptide may include about 1 to about 3 disulfide bonds. Exemplary antimicrobial peptides include, but are not limited to, Indolicidin, Cecropin P1, Dermaseptin, Ponericin W1, Ponericin W3, Ponericin W4, Ponericin W5, Ponericin W6, Maximin H5, Dermcidin, Andropin, Moricin, Cerototoxin, Melittin, Megainin, Bombinin, Brevinin, Esculentin, Buforin, CAP18, LL37, Abaecin, Prophenin, Protegrin, Tachyplesin, Defensin, Drosomycin, Apidaecin, Oncocin, or variants thereof. Additional antimicrobial peptides include those described in U.S. Patent Publication No. 2015/0050717, which is hereby incorporated by reference in its entirety.

In some embodiments, the engineered bacteriophage encodes an antimicrobial peptide selected from an Apidaecin and/or Oncocin. Apidaecins (apidaecin-type peptides) are a series of small, proline-rich (Pro-rich), 18- to 20-residue peptides, which are naturally produced by insects. Structurally, Apidaecins consist of two regions, the conserved (constant) region, responsible for the general antibacterial capacity, and the variable region, responsible for the antibacterial spectrum. The small, gene-encoded and unmodified apidaecins are predominantly active against many Gram-negative bacteria by special antibacterial mechanisms.

In some embodiments, the antimicrobial polypeptide is a lytic enzyme, such as an endolysin, a lysozyme, a lysostaphin, or a functional derivative thereof. These enzymes range in size from 50 to several hundreds of amino acids, and are predominantly used by bacteriophages and bacteria in inter- and intraspecies bacteriocidal warfare. In an embodiment, the enzymes induce the lysis of Gram-positive and/or Gram-negative bacteria. For example, the enzymes may effectively lyse one or more of Staphylococcus aureus, coagulase-negative staphylococci, streptococci, enterococci, anaerobes, and Gram-negative bacilli. Exemplary enzymes include, but are not limited to, LysK, lysozyme, lysostaphin or a functional fragment thereof. In an embodiment, the functional fragment of LysK is CHAP165 as disclosed in U.S. Patent Publication No. 2015/0050717, which is hereby incorporated by reference in its entirety. Additional enzymes are described, for example, in U.S. Patent Publication No. 2015/0050717, which is hereby incorporated by reference in its entirety.

In some embodiments, the bacteriophage is engineered to comprise a nucleic acid encoding a chimera or fusion between the antimicrobial peptide and the lytic enzyme. In certain embodiments, the fusion or chimeric protein may induce the lysis of Staphylococcus aureus and/or other Gram-positive and Gram-negative bacteria. In an embodiment, the fusion or chimeric protein is particularly active against Gram-negative bacteria with an outer membrane. In an embodiment, the fusion or chimeric protein induces the lysis of Staphylococcus aureus which lacks an outer membrane as well as any neighboring Gram-negative bacteria. Exemplary chimeric or fusion proteins between an antimicrobial peptide and a lytic enzyme are described, for example, in U.S. Pat. Nos. 8,096,365 and 8,846,865, and Briers et al., (2015), Future Microbiol, 10(3): 377-90, Briers et al., (2014), Antimicrob Agents Chemother, 58(7): 3774-84, Briers et al., (2014), M. Bio, 5(4): e01379-14, and Lukacik et al., (2012), Proc Natl Acad Sci USA, 109(25): 9857-62, all of which are hereby incorporated by reference in their entireties.

In various embodiments, the bacteriophage is engineered to comprise a nucleic acid encoding an agent that potentiates antibiotic action, for example, by inhibiting the expression and/or function of an antibiotic resistance gene or a cell survival repair gene. Exemplary antibiotic resistance genes to target according to these embodiments are those that confer resistance to beta-lactams (e.g., methicillin) or vancomycin.

Exemplary cell survival repair genes include Staphylococcus orthologs of recA, recB, recC, spoT or relA. Additional targets are disclosed, for example, in U.S. Patent Publication No. 2010/0322903, which is hereby incorporated by reference in its entirety. The expression or function of these genes may be targeted, for example, by expression of antisense polynucleotides, or double stranded RNA or other gene silencing techniques that are functional in the targeted host.

In various embodiments, the bacteriophage is engineered to comprise a nucleic acid encoding at least one gene that represses an SOS response gene and/or a non-SOS pathway bacterial defense gene. The SOS response in bacteria is an inducible DNA repair system, which allows bacteria to survive increased DNA damage. In some embodiments, the repressor is the Staphylococcus ortholog of lexA, or modified version thereof such as lexA3. In some embodiments, the gene represses SOS response genes such as marRAB, arcAB and lexO. Additional repressors are disclosed, for example, in U.S. Patent Publication No. 2010/0322903, which is hereby incorporated by reference in its entirety. In some embodiments, a repressor of a non-SOS pathway gene is one or more of soxR, marR, arc, fur, crp, icdA, craA, or ompA, or modified versions thereof. A non-SOS bacterial defense gene refers to genes expressed by a bacteria or a microorganism that serve to protect the bacteria or microorganism from cell death, for example, from being killed or growth suppressed by an antimicrobial agent.

In various embodiments, the bacteriophage is engineered to comprise a nucleic acid encoding an agent that increases the susceptibility of bacteria to an antimicrobial agent. In one embodiment, the agent increases the entry of an antimicrobial agent into a bacterial cell. Exemplary agents that increase the entry of an antimicrobial agent into a bacterial cell include, but are not limited to genes encoding porin or porin-like proteins, such as OmpF, beta barrel porins, or other members of the outer membrane porin (OMP) functional superfamily. In another embodiment, the agent increases iron-sulfur clusters in the bacteria cell and/or increases oxidative stress or hydroxyl radicals in the bacteria. Examples of a susceptibility agent that increases the iron-sulfur clusters include agents that modulate (i.e. increase or decrease) the Fenton reaction to form hydroxyl radicals. Examples of agents that increase iron-sulfur clusters in the bacterial cell include, for example but not limited to genes encoding the proteins or homologues of IscA, IscR, IscS and IscU. Examples of agents which increase iron uptake and utilization include, for example but not limited to genes encoding the proteins or homologues of, EntC, ExbB, ExbD, FecI, FecR, FepB, FepC, Fes, FhuA, FhuB, FhuC, FhuF, NrdH, NrdI, SodA and TonB. Additional agents that may increase the susceptibility of bacteria to an antimicrobial agent are disclosed, for example, in U.S. Patent Publication No. 2010/0322903, which is hereby incorporated by reference in its entirety.

In various embodiments, the bacteriophage is engineered to comprise a nucleic acid encoding a detectable marker. In an embodiment, the marker is a detectable marker, such as a luminescent or fluorescent protein. Exemplary markers include, for example, luciferase, a modified luciferase protein, blue/UV fluorescent proteins (for example, TagBFP, Azurite, EBFP2, mKalama1, Sirius, Sapphire, and T-Sapphire), cyan fluorescent proteins (for example, ECFP, Cerulean, SCFP3A, mTurquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOK, mKO2, mOrange, and mOrange2), red fluorescent proteins (for example, mRaspberry, mCherry, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatible fluorescent proteins (for example, PA-GFP, PAmCherryl, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), and photoswitchable fluorescent proteins (for example, Dronpa).

In some embodiments the detectable marker comprises a tag. The tag may be used for the detection or the production of the marker. In some embodiments the tag is an affinity tag used to purify and/or concentrate marker. In some embodiments the tag is a 6×His tag. In some embodiments, the tag is an epitope specifically recognized by an antibody that is used to purify and/or concentrate marker produced in the sample prior to detection, and/or that is used to detect the marker. In some embodiments, the detectable marker may comprise a unique nucleic acid sequence that may be amplified (e.g., by polymerase chain reaction (PCR)) to detect the presence of or to quantify the gene encoding the specific marker. Thus any nucleic acid sequence contained within the bacteriophage could be used for PCR-based detection or quantification (e.g., RT-PCR).

In various embodiments, the bacteriophage comprises a promoter sequence operatively linked to direct expression of the genes disclosed herein (for example, nucleic acids encoding a biofilm-degrading enzyme, an antimicrobial polypeptide, an agent that inhibits an antibiotic resistance gene and/or a cell survival repair gene, an agent that increases the susceptibility of a bacteria cell to an antimicrobial agent, and a marker). In some embodiments, the promoter is operatively linked to the nucleic acid. In some embodiments, the promoter is a bacteriophage promoter or a Staphylococcus promoter. Other promoters that may be used are disclosed, for example, in U.S. Patent Publication No. 2010/0322903 and at partsregistry.org/cgi/partsdb/pgroup.cgi?pgroup=other_regulator&show=1, which are hereby incorporated by reference in their entireties.

In various embodiments, the bacteriophage delivers the nucleic acids expressing an agent such as, for example, a biofilm-degrading enzyme and an antimicrobial polypeptide, into the infected host bacterial cell. In an embodiment, the agent is released from the host bacterial cell when the host cell is lysed during the lytic cycle of bacteriophage infection. In another embodiment, the agent is secreted from the host cell, for example, via the secretory pathway. In such an embodiment, the agent which is expressed from the bacteriophage-infected host bacterial cell may contain a signal peptide such as a secretory signal sequence. Such a secretory signal sequence allows intracellular transport of the agent to the bacterial cell plasma membrane for its secretion from the bacteria. Exemplary secretory signal sequences are disclosed, for example, in U.S. Patent Publication No. 2015/0050717, which is hereby incorporated by reference in its entirety.

In one aspect, the present invention provides pharmaceutical compositions comprising one or more bacteriophages of the invention. In some embodiments, the pharmaceutical compositions of the invention may additionally include pharmaceutically acceptable excipient or carrier suitable for application to a site of infection.

The present invention provides methods of treating prosthetic joint infections comprising administering to the infected area and/or the surface of the prosthetic the inventive bacteriophage and/or pharmaceutical composition as disclosed herein. In some embodiments, the bacteriophage and/or pharmaceutical composition effectively inhibit the growth of and/or kill (or reducing the cell viability) the microorganisms (e.g., Staphylococcus aureus) involved with the prosthetic joint infections. In some embodiments, the bacteriophage and/or pharmaceutical composition is effective in eliminating or reducing the bacterial biofilm produced by the microorganisms (e.g., Staphylococcus aureus) involved with the prosthetic joint infections.

In some embodiments, methods of the invention inhibit the growth of and/or kill (or reduce the cell viability) microorganisms in the vicinity of the bacteriophage. In some embodiments, methods of the invention eliminate or reduce bacterial biofilms in the vicinity of the bacteriophage. Without wishing to be bound by theory, it is believed that agents are released into the vicinity from the infected host microbial cell. Accordingly, methods of the invention can target microorganisms involved with prosthetic joint infection even if these microorganisms have not been infected or are resistant to being infected with the bacteriophages of the invention.

In various embodiments, the prosthetic joint infection involves Staphylococcus aureus. In some embodiments, the prosthetic joint infection is a mixed infection involving Staphylococcus aureus and one or more additional microbial species and/or strains. In an embodiment, the additional microbial strain is Gram-positive or Gram-negative. In another embodiment, the additional microbial strain is selected from coagulase-negative staphylococci, streptococci, enterococci, anaerobes, and Gram-negative bacilli. In an embodiment, the additional microbial strain is Staphylococcus epidermidis.

In various embodiments, the bacteriophage or pharmaceutical composition of the invention may be administered in combination with an additional therapeutic agent to a subject in need thereof. In an embodiment, the additional therapeutic agent is an antibiotic or antimicrobial agent, which is administered locally or systemically. In an embodiment, the additional therapeutic agent is an antibiotic or antimicrobial agent which is administered systemically. In various embodiments, administration of the bacteriophage or pharmaceutical composition of the invention in combination with the additional therapeutic agent produces synergistic effects.

Antibiotics suitable for use in the present invention include, but are not limited to, aminoglycosides, carbapenemes, cephalosporins, cephems, glycopeptides fluoroquinolones/quinolones, oxazolidinones, penicillins, streptogramins, sulfonamides rifamycins and/or tetracyclines.

In another aspect, the present invention provides methods for determining the presence or absence of susceptible Staphylococcus aureus in a sample derived from a subject having prosthetic joint infection. The method includes exposing the sample to a GRCS bacteriophage or bacteriophage comprising a minor tail protein as encoded by the GRCS bacteriophage gene 18505614, or a functional derivative thereof. In an embodiment, the bacteriophage includes a nucleic acid encoding a detectable marker, and which is expressed by the host bacteria. The sample is subsequently assayed to detect the presence of absence of the marker, which is indicative of the presence of absence of susceptible Staphylococcus aureus in the sample. Where a sample tests positive for susceptible bacteria, the patient is treated with the bacteriophage described herein.

In various embodiments, the marker is a detectable marker, such as a chemiluminescent or fluorescent protein. Exemplary markers include, for example, luciferase, a modified luciferase protein, blue/UV fluorescent proteins (for example, TagBFP, Azurite, EBFP2, mKalama1, Sirius, Sapphire, and T-Sapphire), cyan fluorescent proteins (for example, ECFP, Cerulean, SCFP3A, mTurquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOK, mKO2, mOrange, and mOrange2), red fluorescent proteins (for example, mRaspberry, mCherry, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatible fluorescent proteins (for example, PA-GFP, PAmCherryl, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), and photoswitchable fluorescent proteins (for example, Dronpa). Methods for the detection of these markers are known in the art and are disclosed for example, in U.S. Patent Publication No. 20150004595m which is hereby incorporated by reference in its entirety

In some embodiments the detectable marker comprises a tag. In some embodiments the tag is a 6×His tag. In some embodiments, the tag is an epitope specifically recognized by an antibody that is used to purify and/or concentrate marker produced in the sample prior to detection, and/or that is used to detect the marker. In some embodiments, the detectable marker may comprise a unique nucleic acid sequence that may be amplified (e.g., by polymerase chain reaction) to identify the presence of or to quantify the gene encoding the specific marker. Any nucleic acid sequence contained within the bacteriophage could be used for PCR-based detection or quantification (e.g., RT-PCR).

In other aspects, the invention provides a method for making GRCS phage from Staphylococcus host cells transformed with a bacterial artificial chromosome (BAC) harboring the GRCS genome. Phage produced with this method are useful for treating infection involving Staphylococcus, including but not limited to PJI. Phage produced with this method can contain one or more gene inserts as described, or other modifications described herein. Such phage can be stabilized with, for example, an osmotic stabilizer. Osmotic stabilizers include, without limitation, saccharides and polymers such as sucrose, trehalose, sorbitol, polyethylene glycol (e.g., MW between 2000 and 10,000), and spermine. In some embodiments, stabilized phage is lyophilized.

BAC vectors replicable in E. coli are well known. After assembly of the GRCS genome into a suitable BAC vector, for example, using Gibson Assembly, the BAC vector containing the phage genome is transformed and purified from E. coli, and introduced into Staphylococcus host cells for production of phage. While many phage contain a terminal protein that is required to initiate DNA replication, it is discovered that while GRCS may contain such a protein associated with phage DNA, the protein is dispensable for DNA replication, allowing convenient production of phage from Staphylococcus host cells transformed with a DNA construct that is replicable in E. coli.

### EXAMPLES

### Example 1: Analysis of Podoviridiae Bacteriophage Genome Sequences

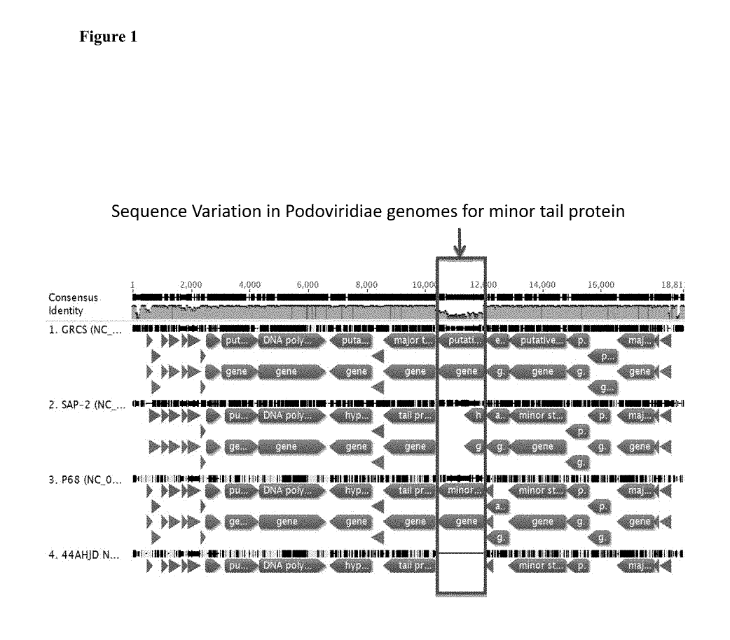

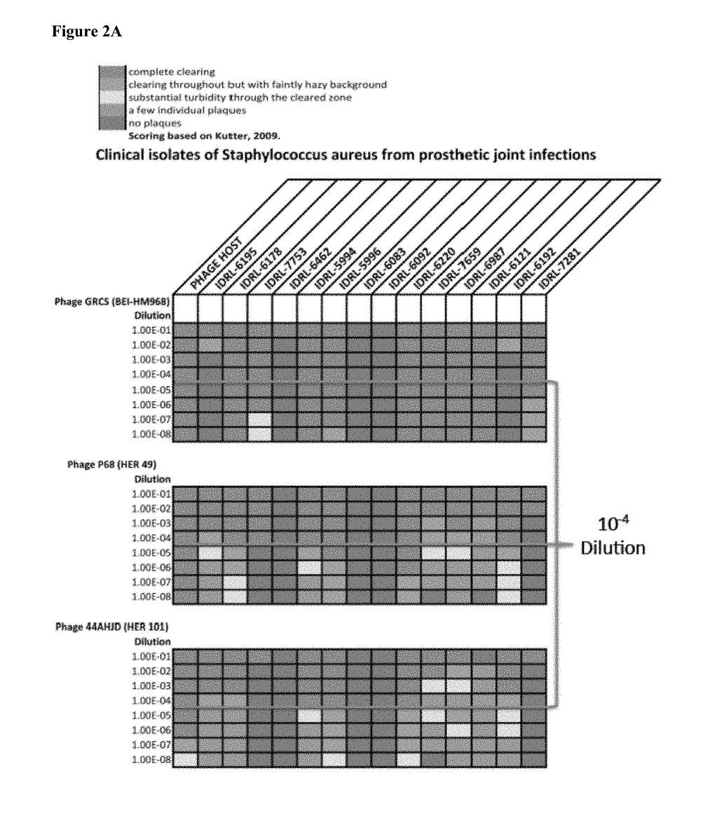

The genome sequences of four Podoviridae bacteriophages GRCS, SAP-2, 44AHJD and P68 were compared using Geneious version 6.1.4. Comparison of the genome sequences across these phages illustrated a high level of homology, indicating a high degree of relatedness to each other. However, genome analysis also revealed significant sequence divergence in a single ORF, i.e., the GRCS gene 18505614 (˜10,000 to 11,500). The GRCS gene 18505614 encodes for a putative minor tail protein, compared to SAP-2 (truncated), P68 (lack of homology) and to 44AHJD (missing open reading frame) (see FIG. 1).

### Example 2: Analysis of Bacteriophage Infection Efficiency of Staphylococcus aureus Isolated from Prosthetic Joint Infection (PJI)

The plaque-forming efficiency of 3 highly related virulent Podoviridae phages was determined using dilution agar overlay assays with S. aureus from 14 non-implant strains (multiple sources) and 27 isolates from PJIs. Lytic efficiency was scored visually on a scale of 4 (total clearing) to 0 (no plaque formation) (Kutter, E. (2009) Methods Mol Biol 501: 141-149).

Phage Propagation

Each phage was propagated on its cognate host in tryptic soy broth supplemented with 10 mM MgSO₄ (TSBM). Phage lysates were prepared by inoculating each designated host with the corresponding phage in liquid TSBM and incubating at 37° C. until lysis was achieved as indicated by the visual clearing of bacteria from each culture. Lysates were then filtered through a 45 μm filter, treated with 50 μl of chloroform per 3 mls of lysate, and stored in a glass vial at 4° C. for future use.

Phage Enumeration by Soft Agar Overlay

A series of soft agar plates were prepared by adding 100 μl of an overnight culture of each host Staphylococcus aureus strain to 3 ml of TSBM+0.75% agar and overlayed onto solid TSBM media (1.5% agar) in a round petri dish. 10-fold dilutions of each phage lysate were simultaneously prepared and a 100 μl volume of each dilution was added to the 3 ml of TSBM+0.75% agar above just prior to its addition to solid TSBM media. Plates were incubated at 37° C. overnight and the number of plaques were visually quantified. Plaque forming units per ml (pfu/ml) were then calculated based on the number of plaques formed and the dilution factor of the lysate inoculum.

Phage Host Range Determination

Staphylococcus aureus cultures were grown in tryptic soy media (Teknova) supplemented with 10 mM Magnesium Sulfate (TSBM). 100 μl of an overnight culture of the indicated S. aureus strain was added to 4 ml of TSBM+0.75% agar and overlayed onto solid TSBM media (1.5% agar) in a square gridded petri dish. Aliquots of each phage with a starting concentration of 10⁸ to 10¹⁰ plaque-forming units (pfu) per ml were serial diluted 10-fold. A 5 μl volume of each dilution was spotted on each bacterial soft-agar overlay. Plates were incubated at 37° C. overnight to allow for plaque formation at each zone of clearing. Zones were scored according to Kutter (Kutter 2009) where a 4 indicated complete clearing, 3 indicated clearing throughout but with faint turbidity through the cleared zone, 2 indicated substantial turbidity, 1 indicated a few individual plaques, and 0 indicated no clearing. Plates were photographed.

Results

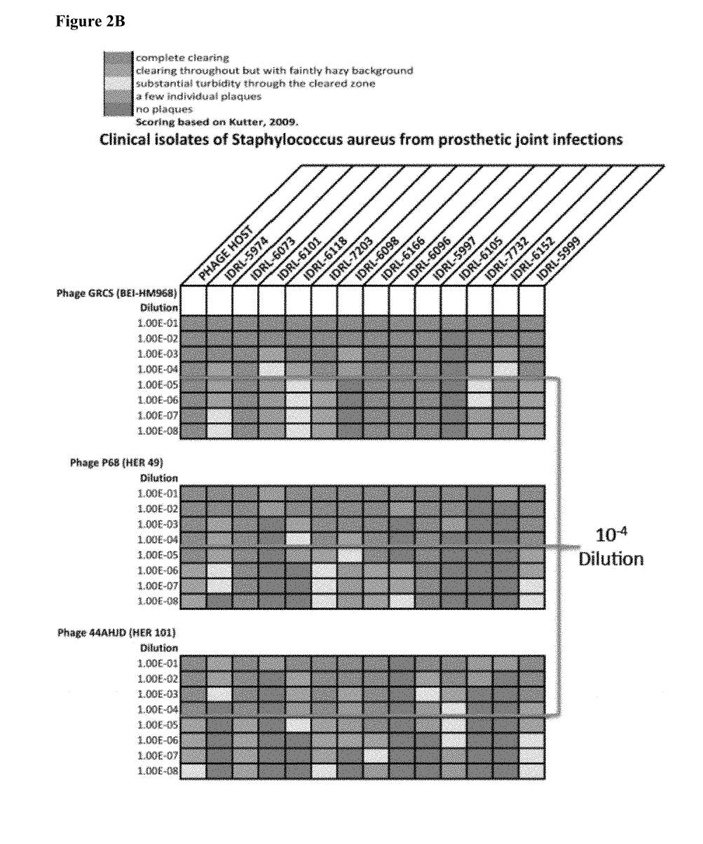

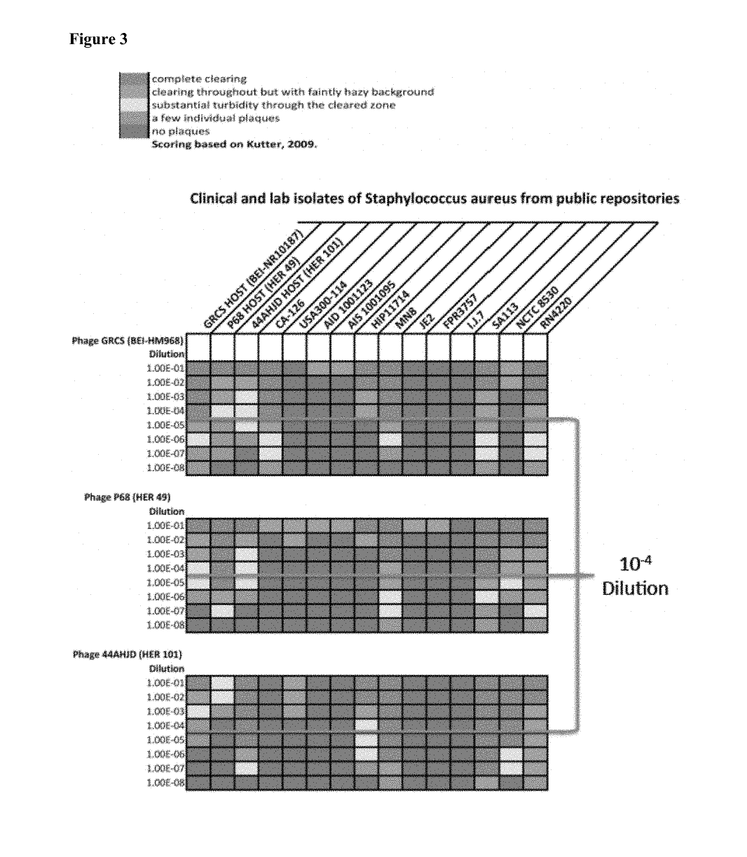

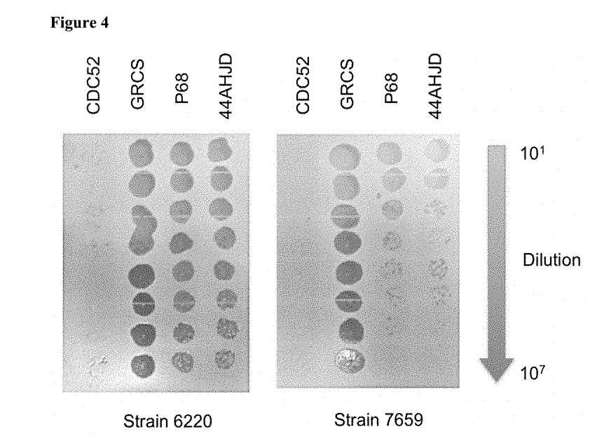

Surprisingly, comparison of the relative infectivity of three bacteriophages (GRCS, P68 and 44AHJD) and others) demonstrated that GRCS has a higher rate of infectivity than the other bacteriophages tested for Staphylococcus aureus (Sau) isolated from PJIs (FIGS. 2A and 2B, FIG. 4, and Table 1), but not when compared against strains from other clinical presentations (FIG. 3).

TABLE 1

| | PJI¹ | Non-implant¹ |
|---|---|---|
| Phage | % strains infected | % strains infected |
| GRCS | 74% | 14% |
| P68 | 48% | 7% |
| 44AHJD | 22% | 21% |

¹Total clearing at 10⁴ dilution (from 10⁹ pfu/ml)

The Podoviridae phages tested demonstrated better plaque-forming efficiency with isolates from PJI as opposed to non-implant strains, except for 44AHJD, which is missing the gene encoding the minor tail protein. The increasing efficiency of phage infection observed for these phages with PJI isolates suggests that strains isolated from PJI may express a determinant, possibly recognized by the minor tail protein in these Podoviridae, phages which is the only significantly divergent sequence in the phage genomes. Phage infectivity of S. aureus may thus depend, in part, on expression and cognate recognition of determinants that appear to be related to clinical source and/or microenvironment of the isolates.

Altogether, these results demonstrated that a diverse set of clinical isolates of S. aureus, from PJIs, were more readily infected by a specific phage (i.e., GRCS) than other strains of S. aureus isolated from other clinical presentations. It was unexpected that in screening related and unrelated bacteriophages against isolates from both PJIs and from other clinical presentations, that one bacteriophage (GRCS) had a greater degree of infectivity (>70%) for strains from PJIs than from other clinical presentations (<40%). Without wishing to be bound by theory, it is believed that this specificity may be due to variations in a specific gene within the GRCS bacteriophage genome, such that highly related bacteriophages (44AHJD and P68) show significant sequence variation from GRCS only in this specific gene and not in other genes across the bacteriophage genomes. These results suggest that many S. aureus strains from PJIs exhibit a specific surface determinant that is specifically recognized by the variant protein in GRCS as opposed to the same functional protein in the other bacteriophages. As such, the GRCS bacteriophage preferentially infects S. aureus strains specifically for the treatment of PJIs.

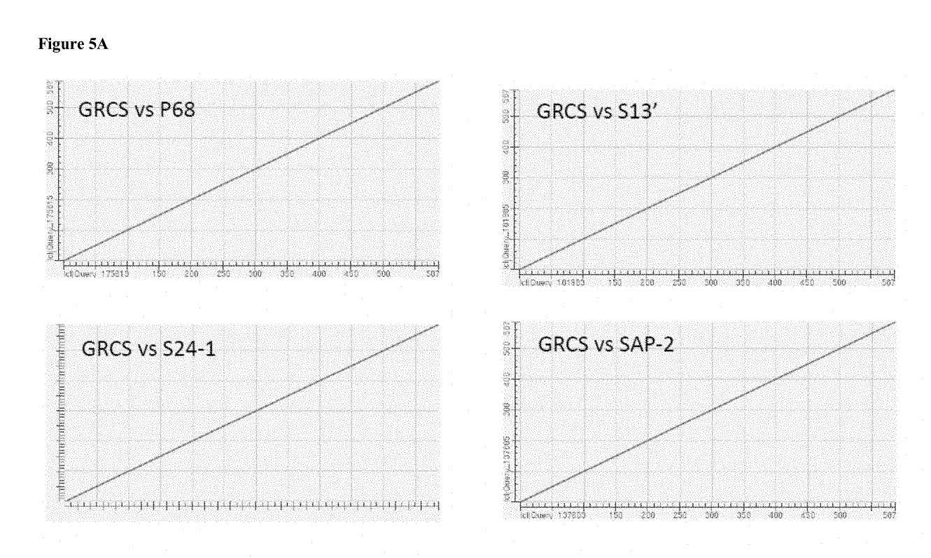

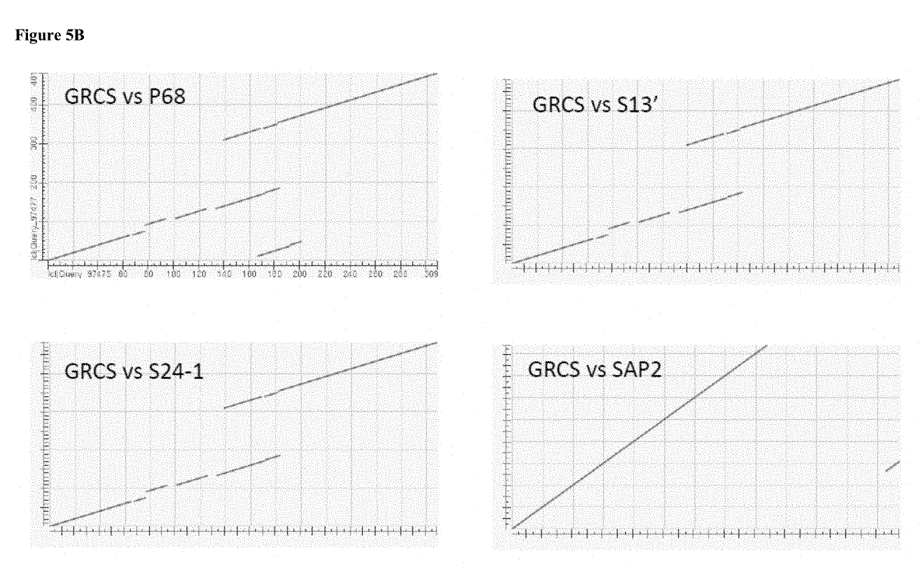

The specificity of GRCS phage for PJI clinical isolates is believed to be at least partly due to the minor tail protein. A dot-matrix homology between the major tail protein of GRCS compared to 3 other Podoviridiae phages (highly related in sequence) shows that the proteins are essentially identical (line is continuous) in all four phages (FIG. 5A). In contrast, as shown in FIG. 5B, the minor tail proteins have varying levels of identity. These are likely the result of gene duplication and deletion. Comparisons between minor tail proteins of related phages may identify regions useful for engineering chimeric minor tail proteins to alter or expand species selectivity.

### Example 3: Cloning of the GRCS Genome into Bacterial Artificial Chromosome

Terminal protein-primed DNA phages have a small protein covalently attached to the 5′ ends of their dsDNA genomes. The terminal protein-primed DNA replication has been found in several phages related to GRCS including P68, a highly homologous S. aureus phage. The literature describes that terminal proteins are required for DNA replication and packaging of these phage genomes. This would putatively prevent insertion of an intact phage genome into a vector for propagation of the phage, as without the terminal protein, replication and packaging would not occur.

Comparisons to known terminal proteins in other phages did not reveal any homologue in the GRCS genome. The DNA polymerase of GRCS does have the signature amino acids found in other protein-primed DNA polymerases from these other phages, suggesting that GRCS utilizes a protein-primed DNA replication mechanism.

Based on the following observations, it was discovered that the terminal protein is dispensable for DNA replication in GRCS. The treatment of GRCS DNA isolated from phage does not enter agarose gels upon electrophoresis, when isolated in the absence of proteinase K treatment to maintain the terminal protein. This is consistent with behavior seen in other phages such as phi29 from Bacillus subtilis, where maintenance of the terminal protein prevents migration of phage genomic DNA in agarose electrophoresis. Treatment with proteinase K during DNA isolation destroys the terminal protein, and allows migration of phage genomic DNA in agarose electrophoresis. Further, both forms of GRCS phage DNA (with terminal protein intact and with the terminal protein destroyed by proteolysis) were transformed directly into the GRCS host strain of S. aureus. Replicating, propagating GRCS phage were obtained only with the proteinase K treated phage genomic DNA. The intact terminal protein DNA did not transform most likely. Importantly, the ability to achieve phage replication and propagation in the absence of the terminal protein shows that the terminal protein is dispensable to initiate DNA replication of the GRCS phage genomic DNA. Accordingly, production from phage in host cells from a bacterial artificial chromosome (BAC) might be successful.

GRCS genomic DNA was isolated from host cell transduction. Briefly, bacterial host (S. aureus) was infected with GRCS phage in liquid culture and allowed to reach lysis (˜4-6 hours). The lysed culture was centrifuged and the supernatant containing GRCS phage particles were harvested by precipitation with PEG followed by purification by CsCl gradient centrifugation. Phage DNA was isolated from the phage particles by disruption of the phage particles with SDS and proteinase K, plus 5 mM EGTA. DNA was isolated by phenol/chloroform extraction and ethanol precipitation.

Overlapping 6 kb segments of the GRCS genome were PCR amplified, followed by purification of the PCR products. The segments contained 20 bp of overlapping DNA sequences for seamless assembly to its neighboring region (based upon the sequence in Vybiral et al., Complete nucleotide sequence and molecular characterization of two lytic Staphylococcus aureus phages: 44AHJD and P68, FEMS Microbiol. Lett. 219, 275-283 (2003)).

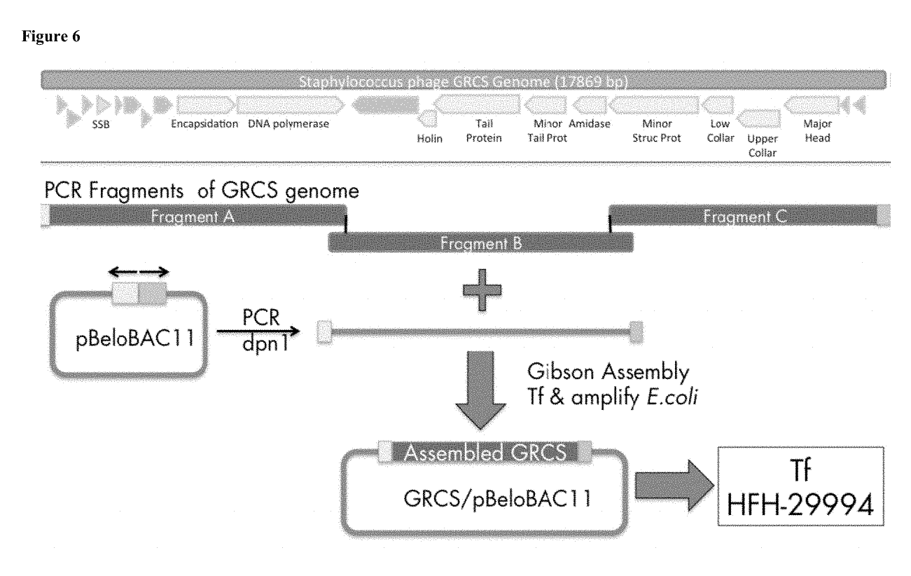

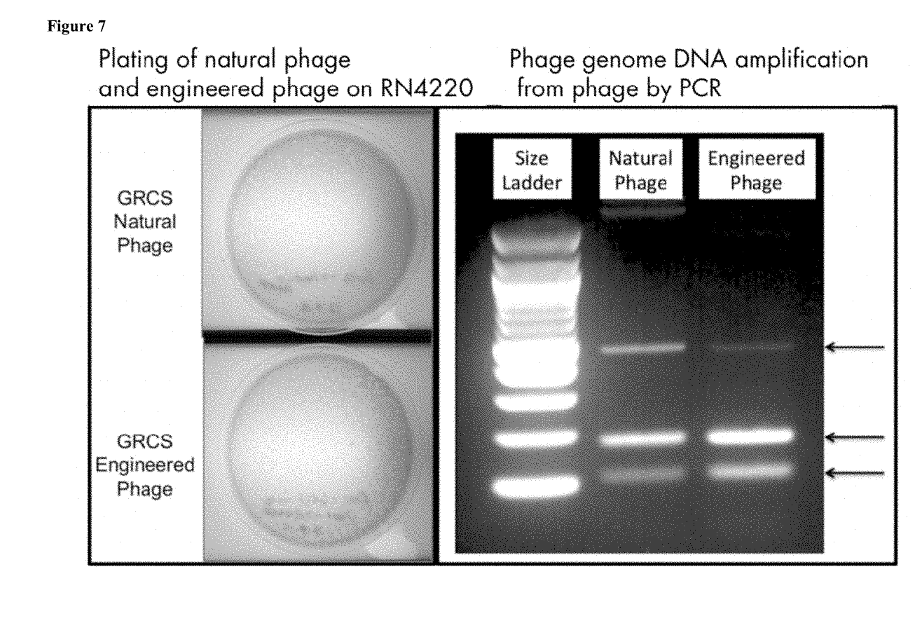

The construction of pBeloBac11-GRCS was conducted by a Gibson Assembly reaction using the NEBuilder® HiFi DNA Assembly Master Mix. See FIG. 6. Four PCR fragments comprising the pBeloBac11 vector backbone and the three˜6 kb segments of the GRCS genome were assembled according to the manufacturer's instructions and the reaction subsequently transformed into electrocompetent DH5-α E. coli. Transformants were obtained by plating cells on LB media containing 20 μg/ml chloramphenicol, which selects for the pBeloBac11 backbone. GRCS genome integration was confirmed by PCR on the purified GRCS-BAC construct. The GRCS-BAC construct was then transformed into the GRCS host strain of S. aureus (Tf HFH-29994 in FIG. 5), allowed to recover from electroporation for 4 hours and then the supernatant of the recovered transformation (after low speed centrifugation) was plated onto a lawn of S. aureus bacteria using soft agar overlay method. Transformation of the GRCS-BAC construct in S. aureus resulted in phage production as determined by plaque formation in the agar overlay, without replication of the vector (the BAC) in the bacteria, as the BAC only replicates in E. coli (FIG. 7). Isolated plaques were checked for the presence of GRCS genomic DNA by PCR and confirmed to be bona fide GRCS phage.

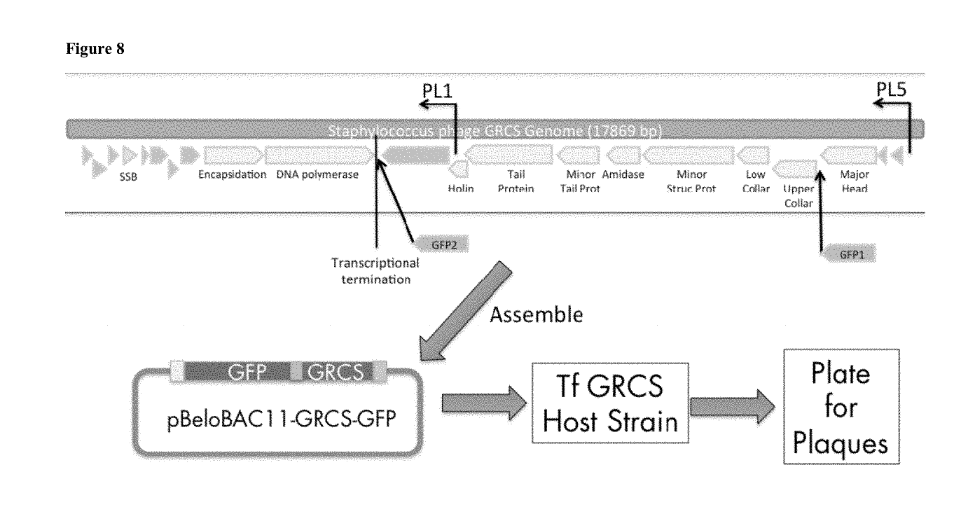

This platform was used to create GFP insertions into the GRCS genome (FIG. 8). Increased osmotic pressure (e.g., with 0.5 M sucrose) was useful in stabilizing phage containing the inserts.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### INCORPORATION BY REFERENCE

All patents and publications referenced herein are hereby incorporated by reference in their entireties.

SEQUENCE LISTING

GRCS bacteriophage gene 18505614 (SEQ ID NO: 1)
atggctgatagaatcgtaagaagtttaaggggtattgattcagtagagaagttaaac
gacaatttagtagaagcaaatgacttaattacaactaaagacgataacatatatata
agacgtgatgaggattattataagttaacctttaaagatgaattattagaaaaaatt
aatacaaacacaaattcaattgataaaaataaaaatgatatcgctacaaataaaaac
aatatatctcaaaatgcaacagatattattcatattaaagaggataatatacaacaa
gataaaaaaattaaaaatttatctgatacacaatcagaacatacaaataaaataaac
aatcacgatgacgctattttgttattagatgatgaaaatacaaaaaacaaattagca
attgaaacgaataaacaagatatcatcgctacaaaagaaacaatcggacaaaataaa
caaagtatagaaaatttagcttcaacggtttcaaacaacacaattgaaacaagtaaa
aaaatcgaatcaactaaaacagaattaatagataaaattaacaattcaaaaacaaat
gtaattgatacaggttggcaagatataacattagaaagtggtattactgcaagtgat
tcaagtggtggttatccttctccgcaataccgtattattacaattaataatattcgt
acaatacaaataagaggagtattaaaaggaattaagaaaaacggagatattaaatta
ggtagtattaatgctaatttaaaaacaacacatcactatacacaatgtgctattgat
acaaaaatgataaatacaagaatgtatttaaattttaacaacgaattacattttgtt
acatcatcgtatcaagatagtgaattaacaaacggtgataaacgttttgtaatagat
acacaaatcattgaataa
Bacteriophage GRCS Putative Minor Tail Protein (SEQ ID NO: 2)
MADRIVRSLRGIDSVEKLNDNLVEANDLITTKDDNIYIRRDEDYYKLTFKDELLEKI
NTNTNSIDKNKNDIATNKNNISQNATDIIHIKEDNIQQDKKIKNLSDTQSEHTNKIN
NHDDAILLLDDENTKNKLAIETNKQDIIATKETIGQNKQSIENLASTVSNNTIETSK
KIESTKTELIDKINNSKTNVIDTGWQDITLESGITASDSSGGYPSPQYRIITINNIR
TIQIRGVLKGIKKNGDIKLGSINANLKTTHHYTQCAIDTKMINTRMYLNFNNELHFV
TSSYQDSELTNGDKRFVIDTQIIE
Dispersin B (Accession No. AY228551) (SEQ ID NO: 3)

| | |
|---|---|
| 1 | aattgttgcg taaaaggcaa ttccatatat ccgcaaaaaa caagtaccaa gcagaccgga |
| 61 | ttaatgctgg acatcgcccg acatttttat tcacccgagg tgattaaatc ctttattgat |
| 121 | accatcagcc tttccggcgg taattttctg cacctgcatt tttccgacca tgaaaactat |
| 181 | gcgatagaaa gccatttact taatcaacgt gcggaaaatg ccgtgcaggg caaagacggt |
| 241 | atttatatta atccttatac cggaaagcca ttcttgagtt atcggcaact tgacgatatc |
| 301 | aaagcctatg ctaaggcaaa aggcattgag ttgattcccg aacttgacag cccgaatcac |
| 361 | atgacggcga tctttaaact ggtgcaaaaa gacagagggg tcaagtacct tcaaggatta |
| 421 | aaatcacgcc aggtagatga tgaaattgat attactaatg ctgacagtat tacttttatg |
| 481 | caatctttaa tgagtgaggt tattgatatt tttggcgaca cgagtcagca ttttcatatt |
| 541 | ggtggcgatg aatttggtta ttctgtggaa agtaatcatg agtttattac gtatgccaat |
| 601 | aaactatcct actttttaga gaaaaaaggg ttgaaaaccc gaatgtggaa tgacggatta |
| 661 | attaaaaata cttttgagca aatcaacccg aatattgaaa ttacttattg gagctatgat |
| 721 | ggcgatacgc aggacaaaaa tgaagctgcc gagcgccgtg atatgcgggt cagtttgccg |
| 781 | gagttgctgg cgaaaggctt tactgtcctg aactataatt cctattatct ttacattgtt |
| 841 | ccgaaagctt caccaacctt ctcgcaagat gccgcctttg ccgccaaaga tgttataaaa |
| 901 | aattgggatc ttggtgtttg ggatggacga aacaccaaaa accgcgtaca aaatactcat |
| 961 | gaaatagccg gcgcagcatt atcgatctgg ggagaagatg caaaagcgct gaaagacgaa |
| 1021 | acaattcaga aaaacacgaa aagtttattg gaagcggtga ttcataagac gaatggggat |
| 1081 | gagtga |

Dispersin B (SEQ ID NO: 4)
NCCVKGNSIYPQKTSTKQTGLMLDIARHFYSPEVIKSFIDTISLSGGNFLHLHFSDHENYAIESHLLNQR
AENAVQGKDGIYINPYTGKPFLSYRQLDDIKAYAKAKGIELIPELDSPNHMTAIFKLVQKDRGVKYLQGL
KSRQVDDEIDITNADSITFMQSLMSEVIDIFGDTSQHFHIGGDEFGYSVESNHEFITYANKLSYFLEKKG
LKTRMWNDGLIKNTFEQINPNIEITYWSYDGDTQDKNEAAERRDMRVSLPELLAKGFTVLNYNSYYLYIV
PKASPTFSQDAAFAAKDVIKNWDLGVWDGRNTKNRVQNTHEIAGAALSIWGEDAKALKDETIQKNTKSLL
EAVIHKTNGDE

### REFERENCES

Kim, C. K., M. J. Karau, K. E. Greenwood-Quaintance, A. Y. Tilahun, C. S. David, J. N. Mandrekar, R. Patel and G. Rajagopalan (2015). “Superantigens in Staphylococcus aureus isolated from prosthetic joint infection.” Diagn Microbiol Infect Dis 81(3): 201-207.
Kurtz, S. M., E. Lau, H. Watson, J. K. Schmier and J. Parvizi (2012). “Economic burden of periprosthetic joint infection in the United States.” J Arthroplasty 27(8 Suppl): 61-65 e61.
Kwan, T., J. Liu, M. DuBow, P. Gros and J. Pelletier (2005). “The complete genomes and proteomes of 27 Staphylococcus aureus bacteriophages.” Proc Natl Acad Sci USA 102(14): 5174-5179.
O'Flaherty, S., A. Coffey, R. Edwards, W. Meaney, G. F. Fitzgerald and R. P. Ross (2004). “Genome of staphylococcal phage K: a new lineage of Myoviridae infecting gram-positive bacteria with a low G+C content.” J Bacteriol 186(9): 2862-2871.
O'Flaherty, S., R. P. Ross, W. Meaney, G. F. Fitzgerald, M. F. Elbreki and A. Coffey (2005). “Potential of the polyvalent anti-Staphylococcus bacteriophage K for control of antibiotic-resistant staphylococci from hospitals.” Appl Environ Microbiol 71(4): 1836-1842.

Son, J. S., S. J. Lee, S. Y. Jun, S. J. Yoon, S. H. Kang, H. R. Paik, J. O. Kang and Y. J. Choi (2010). “Antibacterial and biofilm removal activity of a podoviridae Staphylococcus aureus bacteriophage SAP-2 and a derived recombinant cell-wall-degrading enzyme.” Applied microbiology and biotechnology 86(5): 1439-1449.
Tande, A. J. and R. Patel (2014). “Prosthetic joint infection.” Clin Microbiol Rev 27(2): 302-345.
Vybiral, D., M. Takac, M. Loessner, A. Witte, U. von Ahsen and U. Blasi (2003). “Complete nucleotide sequence and molecular characterization of two lytic Staphylococcus aureus phages: 44AHJD and P68.” FEMS Microbiol Lett 219(2): 275-283.
Yilmaz, C., M. Colak, B. C. Yilmaz, G. Ersoz, M. Kutateladze and M. Gozlugol (2013). “Bacteriophage therapy in implant-related infections: an experimental study.” J Bone Joint Surg Am 95(2): 117-125.
Sunagar, R., S. A. Patil and R. K. Chandrakanth (2010). “Bacteriophage therapy for Staphylococcus aureus bacteremia in streptozotocin-induced diabetic mice.” Res Microbiol 161(10): 854-860.
Swift, S. M. and D. C. Nelson (2014). “Complete Genome Sequence of Staphylococcus aureus Phage GRCS.” Genome Announc 2(2).
Briers, Y. and R. Lavigne. “Breaking barriers: expansion of the use of endolysins as novel antibacterials against Gram-negative bacteria.” Future Microbiol, 2015. 10(3): p. 377-90.
Briers, Y., et al. “Art-175 is a highly efficient antibacterial against multidrug-resistant strains and persisters of Pseudomonas aeruginosa.” Antimicrob Agents Chemother, 2014. 58(7): p. 3774-84.
Briers, Y., et al. “Engineered endolysin-based “Artilysins” to combat multidrug-resistant gram-negative pathogens.” MBio, 2014. 5(4): p. e01379-14.
Lukacik, P., et al. “Structural engineering of a phage lysin that targets gram-negative pathogens.” Proc Natl Acad Sci USA, 2012. 109(25): p. 9857-62.
Bjornisti, M. A., Reilly, B. E. & Anderson, D. L. “In vitro assembly of the Bacillus subtilis bacteriophage phi 29.” Proc Natl Acad Sci USA 78, 5861-5865 (1981).
Bjomisti, M. A., Reilly, B. E. & Anderson, D L. “Morphogenesis of bacteriophage phi 29 of Bacillus subtilis: DNA-gp3 intermediate in in vivo and in vitro assembly.” J. Virol. 41, 508-517 (1982).
Blanco, L. et al. “Highly efficient DNA synthesis by the phage phi 29 DNA polymerase. Symmetrical mode of DNA replication.” J. Biol. Chem. 264, 8935-8940 (1989).
Blanco, L & Salas, M. “Replication of phage phi 29 DNA with purified terminal protein and DNA polymerase: synthesis of full-length phi 29 DNA.” Proc. Natl. Acad. Sci USA 82, 6404-6408 (1985).
Ito, J. “Bacteriophage phi29 terminal protein: its association with the 5′ termini of the phi29 genome.” J. Virol. 28, 895-904 (1978).
Gibson, D. G. “Synthesis of DNA fragments in yeast by one-step assembly of overlapping oligonucleotides.” Nucleic Acids Res. 37, 6984-6990 (2009).
Gibson, D. G., et al. “Creation of a bacterial cell controlled by a chemically synthesized genome.” Science 329, 52-56 (2010).
Gibson, D. G., et al. “Enzymatic assembly of DNA molecules up to several hundred kilobases.” Nat. Methods 6, 343-345 (2009).

1. A method for treating prosthetic joint infection involving Staphylococcus aureus, comprising administering to the infected area and/or surface of the prosthetic a GRCS bacteriophage or a Podoviridae bacteriophage having a minor tail protein encoded by GRCS bacteriophage gene 18505614, or a functional derivative thereof.

2. The method of claim 1, wherein the functional derivative comprises an amino acid sequence having at least 70% identity with the minor tail protein encoded by GRCS bacteriophage gene 18505614.

3. The method of claim 1, wherein the minor tail protein or the functional derivative thereof recognizes a surface determinant on Staphylococcus aureus.

4. The method of claim 1, wherein the prosthetic joint infection is a mixed infection comprising one or more additional microbial strains.

5. The method of claim 4, wherein the additional microbial strain is Gram-positive or Gram-negative.

6. (canceled)

7. The method of claim 1, wherein the bacteriophage eliminates the Staphylococcus aureus.

8. (canceled)

9. The method of claim 1, wherein the bacteriophage comprises a nucleic acid encoding a biofilm-degrading enzyme.

10. (canceled)

11. The method of claim 1, wherein the bacteriophage comprises a nucleic acid encoding at least one antimicrobial polypeptide.

12.-18. (canceled)

19. The method of claim 1, wherein the bacteriophage comprises a nucleic acid encoding at least one agent that inhibits an antibiotic resistance gene and/or a cell survival repair gene.

20. The method of claim 1, wherein the bacteriophage comprises a nucleic acid encoding at least one repressor of a SOS response gene and/or bacterial defense gene.

21.-23. (canceled)

24. The method of claim 1, wherein the bacteriophage comprises a nucleic acid encoding at least one agent which increases the susceptibility of a bacteria cell to an antimicrobial agent.

25.-27. (canceled)

28. An engineered bacteriophage comprising a minor tail protein as encoded by GRCS bacteriophage gene 18505614, or a functional derivative thereof,
wherein the minor tail protein or the functional derivative thereof, recognizes a surface determinant on Staphylococcus aureus associated with prosthetic joint infection.

29. The bacteriophage of claim 28, wherein the bacteriophage is engineered to comprise a nucleic acid encoding a biofilm-degrading enzyme.

30. (canceled)

31. The bacteriophage of claim 28, wherein the bacteriophage is engineered to comprise a nucleic acid encoding at least one antimicrobial polypeptide.

32.-38. (canceled)

39. The bacteriophage of claim 28, wherein the bacteriophage is engineered to comprise a nucleic acid encoding at least one agent that inhibits an antibiotic resistance gene and/or a cell survival repair gene.

40. The bacteriophage of claim 28, wherein the bacteriophage is engineered to comprise a nucleic acid encoding at least one repressor of a SOS response gene and/or bacterial defense gene.

41.-43. (canceled)

44. The bacteriophage of claim 28, wherein the bacteriophage is engineered to comprise a nucleic acid encoding at least one agent which increases the susceptibility of a bacteria cell to an antimicrobial agent.

45.-52. (canceled)

53. A pharmaceutical composition comprising a bacteriophage of claim 28, and a pharmaceutically acceptable carrier.

54. The pharmaceutical composition of claim 53, wherein the pharmaceutical composition eliminates Staphylococcus aureus associated with the prosthetic joint infection.

55. The pharmaceutical composition of claim 53, wherein the pharmaceutical composition removes biofilms associated with the prosthetic joint infection.

56.-68. (canceled)


SEQUENCE LISTING (4 sequences)

SEQ ID NO: 1 - DNA, 930 bp, Bacteriophage GRCS
atggctgatagaatcgtaagaagtttaaggggtattgattcagtagagaagttaaacgacaatttagtagaagcaaatgacttaattacaactaaagacgataacatatatataagacgtgatgaggattattataagttaacctttaaagatgaattattagaaaaaattaatacaaacacaaattcaattgataaaaataaaaatgatatcgctacaaataaaaacaatatatctcaaaatgcaacagatattattcatattaaagaggataatatacaacaagataaaaaaattaaaaatttatctgatacacaatcagaacatacaaataaaataaacaatcacgatgacgctattttgttattagatgatgaaaatacaaaaaacaaattagcaattgaaacgaataaacaagatatcatcgctacaaaagaaacaatcggacaaaataaacaaagtatagaaaatttagcttcaacggtttcaaacaacacaattgaaacaagtaaaaaaatcgaatcaactaaaacagaattaatagataaaattaacaattcaaaaacaaatgtaattgatacaggttggcaagatataacattagaaagtggtattactgcaagtgattcaagtggtggttatccttctccgcaataccgtattattacaattaataatattcgtacaatacaaataagaggagtattaaaaggaattaagaaaaacggagatattaaattaggtagtattaatgctaatttaaaaacaacacatcactatacacaatgtgctattgatacaaaaatgataaatacaagaatgtatttaaattttaacaacgaattacattttgttacatcatcgtatcaagatagtgaattaacaaacggtgataaacgttttgtaatagatacacaaatcattgaataa

SEQ ID NO: 2 - protein, 309 aa, Bacteriophage GRCS
MADRIVRSLRGIDSVEKLNDNLVEANDLITTKDDNIYIRRDEDYYKLTFKDELLEKINTNTNSIDKNKNDIATNKNNISQNATDIIHIKEDNIQQDKKIKNLSDTQSEHTNKINNHDDAILLLDDENTKNKLAIETNKQDIIATKETIGQNKQSIENLASTVSNNTIETSKKIESTKTELIDKINNSKTNVIDTGWQDITLESGITASDSSGGYPSPQYRIITINNIRTIQIRGVLKGIKKNGDIKLGSINANLKTTHHYTQCAIDTKMINTRMYLNFNNELHFVTSSYQDSELTNGDKRFVIDTQIIE

SEQ ID NO: 3 - DNA, 1086 bp, Aggregatibacter actinomycetemcomitans
aattgttgcgtaaaaggcaattccatatatccgcaaaaaacaagtaccaagcagaccggattaatgctggacatcgcccgacatttttattcacccgaggtgattaaatcctttattgataccatcagcctttccggcggtaattttctgcacctgcatttttccgaccatgaaaactatgcgatagaaagccatttacttaatcaacgtgcggaaaatgccgtgcagggcaaagacggtatttatattaatccttataccggaaagccattcttgagttatcggcaacttgacgatatcaaagcctatgctaaggcaaaaggcattgagttgattcccgaacttgacagcccgaatcacatgacggcgatctttaaactggtgcaaaaagacagaggggtcaagtaccttcaaggattaaaatcacgccaggtagatgatgaaattgatattactaatgctgacagtattacttttatgcaatctttaatgagtgaggttattgatatttttggcgacacgagtcagcattttcatattggtggcgatgaatttggttattctgtggaaagtaatcatgagtttattacgtatgccaataaactatcctactttttagagaaaaaagggttgaaaacccgaatgtggaatgacggattaattaaaaatacttttgagcaaatcaacccgaatattgaaattacttattggagctatgatggcgatacgcaggacaaaaatgaagctgccgagcgccgtgatatgcgggtcagtttgccggagttgctggcgaaaggctttactgtcctgaactataattcctattatctttacattgttccgaaagcttcaccaaccttctcgcaagatgccgcctttgccgccaaagatgttataaaaaattgggatcttggtgtttgggatggacgaaacaccaaaaaccgcgtacaaaatactcatgaaatagccggcgcagcattatcgatctggggagaagatgcaaaagcgctgaaagacgaaacaattcagaaaaacacgaaaagtttattggaagcggtgattcataagacgaatggggatgagtga

SEQ ID NO: 4 - protein, 361 aa, Aggregatibacter actinomycetemcomitans
NCCVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE